(19) United States
(12) Patent Application Publication
(10) Pub. No.: US 2012/0237628 A1
(43) Pub. Date: Sep. 20, 2012

(54) COMPOSITION FOR PROMOTING HEALTH OF CARDIOVASCULAR SYSTEM

(76) Inventors: Kah Hwi Kim, Kuala Lumpur (MY); Swee Hung Cheah, Kuala Lumpur (MY); Jing Jing Sum, Kuala Lumpur (MY)

(21) Appl. No.: 13/508,216

(22) PCT Filed: Nov. 4, 2010
(86) PCT No.: PCT/MY2010/000251

§ 371 Date: Jun. 6, 2012

(30) Foreign Application Priority Data
Nov. 6, 2009 (MY) PI 20094699

(51) Int. Cl. A61K 36/21; A61P 3/06

(52) U.S. Cl. 424/774; 424/725

(57) ABSTRACT

A composition for lowering plasma triglycerides level and/or increasing plasma high density lipoprotein level upon administration towards a subject comprises a polar extract of plant parts of Alternanthera sessilis.

### FIELD OF INVENTION

The present invention relates to a composition capable of lowering plasma level of triglycerides a subject upon administrating the mentioned composition to the subject. Apart from that, plasma high density lipoprotein (HDL) is increased also in the subject when the subject has taken the composition at an appropriate dose for a period of time. In more specific, the composition contains an extract of plant parts of the plant Alternanthera sessilis, particularly from leaves and/or foliages.

### BACKGROUND OF THE INVENTION

Herbs have been used as food and for medicinal purposes for centuries. Herbal medicine is based on the premise that herbs and herbal extracts contain natural substances that can promote health and provide therapeutic effects. The World Health Organization estimated that approximately 80% of the earth's populations rely on traditional medicine for their primary health care needs, and most of this therapy involves the use of plant extracts or their active components. There are many investigations that are being conducted on various medicine herbs to identify their active compounds that may have beneficial effects towards human health. In spite of limited side effects of traditional medicine herbs, some may be toxic even consumed in small amounts and some may become toxic at higher dose.

In many places in the world, the leaves of Alternanthera sessilis are eaten as cooked vegetable or raw as a salad. It is also been cooked in soup, in sauces, as potherb and so on. The herb is said to have positive effect towards human health. Alternanthera sessilis has been widely used as a medicinal herb in many countries. In Sri Lanka, the herb is used to treat stomach disorder, as a plaster for wounded or diseased skin parts and against fever. In Ghana, a decoction of the Alternanthera sessilis with some salts is taken to stop vomiting blood. The herb is used to treat headache, vertigo and neuralgia in Nigeria. Alternanthera sessilis is used as cholagogue, abortifacient and febrifuge in India. Besides that, the powder form of the herb is applied against snakebites in the country.

Besides, United States patent publication no. 2008102045 discloses a topical preparation containing a natural extracts of broccoli, green tea, and Alternanthera sessilis. The topical preparation mentioned includes sunscreen, UV protection and other similar cosmetic products, while the natural extract functions as antioxidants in the described products.

Further in Japanese patent publication no. 2007051163, extracts of Alternanthera sessilis and other plants are used as active ingredients in preparing cosmetic, food or drink products. The disclosed preparation claims that these active ingredients shall promote collagen production in the users upon significant consumption. Subsequently, skin health of the user is aesthetically enhanced.

### SUMMARY OF THE INVENTION

The present invention aims to provide a composition containing an extract of Alternanthera sessilis which can be health promoting upon oral consumption. Particularly, the extract in the given composition is hydrophilic or polar extract that it can reduce triglycerides level in plasma of a subject. Consequently, it is possible to avoid development of arteriosclerosis in the subject when the composition is frequently consumed.

Further object of the present invention aims to promote health of the cardiovascular system in a subject through using the disclosed polar extract of Alternanthera sessilis. It was found by the inventor that the present invention also capable of increase HDL in plasma level which in turn further prevent on set of arteriosclerosis.

At least one of the preceding objects is met, in whole or in part, by the present invention, in which one of the embodiment of the present invention includes a composition for lowering plasma triglycerides level upon administration towards a subject comprising a polar extract of plant parts of Alternanthera sessilis.

In order to carry out the triglycerides lowering effect in a subject, it is preferred that the administration of the disclosed composition is via oral feeding.

In another aspect, the polar extract is extracted via a solvent selected from the group consisting of water, methanol, isopropanol, acetonitrile and mixtures thereof.

Further embodiment of the present invention also claims the use of polar extract of the plant Alternanthera sessilis for lowering plasma triglycerides level upon administration orally towards a subject.

### DETAILED DESCRIPTION OF THE INVENTION

One skilled in the art will readily appreciate that the present invention is well adapted to carry out the objects and obtain the ends and advantages mentioned, as well as those inherent therein. The embodiment describes herein is not intended as limitations on the scope of the invention.

One of the embodiments of the present invention includes a composition for lowering plasma triglycerides level and/or increasing plasma HDL level upon administration towards a subject comprising a polar extract of plant parts of Alternanthera sessilis. It was found by the inventors of the present invention that taking effective dose of the disclosed composition by the subject can greatly reduce triglycerides level in the plasma. The effective dose refers herein can be varied according to the prepared formulation of the disclosed composition, age or sex of the subject, types of carrier used in the composition and so on. Though the effective mechanism for lowering the triglycerides level in the plasma is unknown, patentability of the present invention shall not be diminished.

According to the preferred embodiment, the present invention is effective in achieving the triglyceride lowering effect or HDL promoting effect via oral ingestion. It is important to be noted that it is possible metabolites in the polar extract of the plant parts of Alternanthera sessilis requires to be firstly converted to its active form through gastrol intestinal digestion. To enhance bioavailability of the disclosed composition, preferably, a suitable carrier is used to promote the absorption upon ingestion of the disclosed composition.

Pursuant to another preferred embodiment, the above mentioned plant parts are leaves and/or foliages. Preferably, these plant parts are dried before subjecting to extraction. In more specific, the dried plant parts are ground into powder form or small aggregates to facilitate the extraction in the present invention. To perform as the triglycerides lowering agent, it was found the polar or hydrophilic extract of Alternanthera sessilis is much effective than the non-polar extract. In order to acquire the polar extract which contains the desired metabolites, a polar solvent is used in the present invention for extraction. Preferably, the solvent is selected from the group consisting of water, methanol, isopropanol, acetonitrile and mixtures thereof.

Another embodiment of the present invention includes use of polar extract of the plant Alternanthera sessilis for lowering plasma triglycerides level upon administration orally towards a subject. Further, use of polar extract of the plant Alternanthera sessilis for increasing plasma HDL level upon administration orally towards the subject is disclosed in the present invention as well. As setting forth, the amount to be administered to the subject can be varied considering the formulation or other factors as above mentioned. Similarly, the polar extract to be used is extracted via a solvent selected from the group consisting of water, methanol, isopropanol, acetonitrile and mixtures thereof.

The following example is intended to further illustrate the invention, without any intent for the invention to be limited to the specific embodiments described therein.

### EXAMPLE 1

Alternanthera sessilis was collected from Herbal Garden, Gunung Ledang, Johor. The whole plant was used in the preparation of the Alternanthera sessilis extraction, including the stems, the roots, the leaves and the flowers.

The whole plant was dried in the oven for 3-4 days at a temperature of 40° C. After that, the dried red tea was ground into powder form by using an electrical blender. The Alternanthera sessilis powder was then been soaked in 95% ethanol for three days at room temperature. Within these three days, the Alternanthera sessilis powder was stirred occasionally. After three days, the suspension was filtered using filter papers. Subsequently, the ethanol was evaporated by rotary evaporator and the Alternanthera sessilis extract was obtained. Finally, the extraction was freeze-dried at −80° C. to remove the water. The freeze-dried Alternanthera sessilis extract was stored in the freezer at temperature of −5° C. until it is used.

### EXAMPLE 2

White rabbits were obtained from Animal Centre of Faculty of Medicine, University of Malaya. Six white rabbits weighing 2.3 kg-2.9 kg which consisted of 3 males and 3 females were used for this study. They were acclimatized to the facility by being individually housed in metal cages with 12 hours light and 12 hours dark cycle and 23° C.-30° C. temperature. They were fed with normal cholesterol-free diet and water ad-libitum. The whole blood cholesterol level of each rabbit was measured in this acclimatization period. The rabbits were then divided randomly into two groups where 3 rabbits in the control group and 3 rabbits in the treatment group.

The induction period for atherosclerosis was carried out for 4 months. Within these 4 months, each rabbit from both control and treatment groups was injected subcutaneously with 95% cholesterol at a dosage of 150 mg/kg three times a week (Monday, Wednesday and Friday) and the rabbits were fed orally with pure cholesterol at a dosage of 100 mg/kg twice a week (Tuesday and Thursday). Both 95% and pure cholesterol were prepared by dissolving 150 mg of cholesterol powder in 1 ml of coconut oil. Besides, the rabbits were given high cholesterol diet which was prepared by mixing 2 kg of ground pellets with 30 egg yolks and 454 g butter manufactured by Fontero of New Zealand (imported by Fontero Band Malaysia Sdn. Bhd.). The cholesterol diet was then dried in an oven at 60° C. for a day. The body weight and the blood cholesterol levels of the rabbits were measured every week.

### EXAMPLE 3

During the first 4 weeks of treatment period, the rabbits in both control and treatment groups were continued with the cholesterol administration which were subcutaneous injection of 95% cholesterol (150 mg/kg) three times a week (Monday, Wednesday and Friday) and oral feeding of pure cholesterol (100 mg/kg) twice a week (Tuesday and Thursday) and high cholesterol diet. Rabbits in the treatment group were given twice daily oral feeding of 100 mg/kg Alternanthera sessilis ethanolic extract solution.

But at the 5^{th} and 6^{th} week of the treatment period, subcutaneous injection and oral feeding of cholesterol and high cholesterol diet were stopped for both groups. Distilled water was given to rabbit in control group and treatment with 100 mg/kg of Alternanthera sessilis ethanolic extract was continued in the rabbits in treatment group.

Table 1 shows the body weight of experimental rabbits prior to the atherosclerosis induction period and at the end of the experiment. Prior to the induction period, the body weight for control group was 2.60±0.17 kg whereas for treatment group was 2.63±0.18 kg. At the end of the treatment period, the weight of rabbits in both groups increased to 3.2 kg in control group and 3.27±0.23 kg in treatment group. The results obtained show that the body weight of rabbits in both control and treatment groups increased significantly at the end of the experiment as compare to body weight prior to atherosclerosis induction. There is no significance difference in the weight of the experimental rabbits were observed between the control group and treatment group during the experimental period.

TABLE 1

| | |
|---|---|
| Weight of | |
| Rabbit | Group |

| (kg/rabbit) | Control Group | Treatment Group |
|---|---|---|
| Prior to the Induction | 2.60 ± 0.17 | 2.63 ± 0.18 |
| Period | | |
| End of the Treatment | 3.2 | 3.27 ± 0.23 |
| Period | | |

Values are mean ± S.E.M.
Groups are as follows: (a) at the beginning of the induction period: control (n = 3) and treatment (n = 3) (b) at the end of the treatment period: control (n = 1) and treatment (n = 3)

### EXAMPLE 4

The blood cholesterol levels of rabbits were tested weekly during the induction period.

During the 6-week treatment period, blood cholesterol and triglyceride levels in the rabbits were measured weekly. Besides, the HDL level was also measured at the end of the treatment period. The blood sample was obtained from the marginal ear vein of the rabbits. The blood was then measured by Reflotron Analyzer with Reflotron cholesterol strips, triglyceride strips and HDL strips. The measurable range for cholesterol is 100 mg/dl-500 mg/dl, 70 mg/dl-600 mg/dl for triglyceride and 10 mg/dl-100 mg/dl for HDL.

Table 2 shows the total blood cholesterol level of the experimental rabbits during the study. At the beginning of the study (prior to atherosclerosis induction), the total blood cholesterol level of the rabbits in both control group and treatment group were less than 100 mg/dl. This indicated that all the rabbits were free of hypercholesterolemia. After that, the experimental rabbits were subcutaneously injected and oral fed with cholesterol and given high cholesterol diet for 4 months to induce atherosclerosis to investigate the possible anti-atherosclerosis effect of the Alternanthera sessilis ethanolic extract. After 4 months of atherosclerosis induction period, the total blood cholesterol levels increased significantly in both control and treatment groups to more than 500 mg/dl. The rabbits now suffered from hypercholesterolemia which is the main characteristics for formation of atherosclerotic plague. After 6 weeks of treatment period, the total blood cholesterol level in control group and treatment group treated with Alternanthera sessilis ethanolic extract still remained above 500 mg/dl. The results showed that oral feeding of Alternanthera sessilis ethanolic extract did not reduce the blood cholesterol level in treated rabbits.

TABLE 2

| | |
|---|---|
| Blood Cholesterol | Groups |

| Level (mg/dl) | Control | Treatment |
|---|---|---|
| Prior to Atherosclerosis | <100 | <100 |
| Induction Period | | |
| End of the Atherosclerosis | >500 | >500 |
| Induction Period | | |
| End of the Treatment | >500 | >500 |
| Period | | |

Groups are as follows: (a) at the beginning of the induction period: control (n = 3) and treatment (n = 3) (b) at the end of the treatment period: control (n = 1) and treatment (n = 3)

In this study, the results show that oral feeding of Alternanthera sessilis ethanolic extract (100 mg/kg, twice per day) for 6 weeks did not change the total blood cholesterol level in the treatment group rabbits. The total blood cholesterol level for both control and treatment groups after treatment period remained at more than 500 mg/dl. This is due to the instrument used to detect the total blood cholesterol level in this study was unable to measure the cholesterol level above 500 mg/dl. Even though there are possible changes in the cholesterol level after oral feeding of Alternanthera sessilis were not able to be detected by the instrument.

Table 3 shows the triglyceride level of the control and treatment groups thorough the treatment period. At the end of the atherosclerosis induction period or beginning of the treatment period, the triglyceride level of rabbit in control group was 413 mg/dl whereas the triglyceride level of rabbits in treatment group was more than 600 mg/dl. After 6 weeks of treatment period, the triglyceride level of rabbit in control group which was fed orally by saline twice daily decreased to 139 mg/dl (decreased by 66.4%). As for the treatment group which was given Alternanthera sessilis ethanolic extract twice daily, the triglyceride level was much decreased to 113.47±14.65 mg/dl (decreased by 82%). The percentage of the reduction in triglyceride level in treatment group was slightly higher as compare to control group.

TABLE 3

| | |
|---|---|
| Triglyceride Level | Group |

| (mg/dl) | Control | Treatment |
|---|---|---|
| End of the Induction | 413 | 600 |
| Period | (100%) | (100%) |
| End of the Treatment | 139 | 113.47 ± 14.65 |
| Period | (33.6%) | (18.0%) |

Values are mean ± S.E.M.
Groups are as follows: control group (n = 1) and treatment group (n = 3)

Table 4 shows the HDL level of the control and treatment groups at the end of the treatment period. The HDL level of control group was less than 10 mg/dl at the end of the study. While the HDL level of treatment group was at much higher value which was 15.9±4.1 mg/dl.

TABLE 4

| Group | Control | Treatment |
|---|---|---|
| HDL Level (mg/dl) | <10 | 15.9 ± 4.1 |

Values are mean ± S.E.M.
Groups are as follows: control group (n = 1) and treatment group (n = 3)

However, the results of this study show that treatment with Alternanthera sessilis ethanolic extract caused much higher decrease in the triglyceride level in treatment group by 82% as compared to the control group whereby the reduction of triglyceride level was only by 66.4%. Hypertriglyceridemia is closely related to CVD includes coronary artery disease and atherosclerosis. Hence, through the ability to reduce the triglyceride level in treatment group, Alternanthera sessilis is said to have potential protective effect towards atherosclerosis.

Besides, HDL level in the treatment group at the end of the study is slightly higher compared to HDL level in the control group (control group: <10 mg/dl; treatment group: 15.9±4.1 mg/dl). Since HDL is good cholesterol in the body system and is protective against atherosclerosis, the higher the HDL level, the lower the risk of getting atherosclerosis.

In this study, cholesterol was administered into the experimental rabbits by two different methods which were subcutaneous injection and oral feeding. Cholesterol was injected subcutaneously into the rabbits since cholesterol is a non-polar compound and hence it can easily diffuse through the membrane into the blood circulation. As for oral feeding, the cholesterol that was directly fed into the gastrointestinal tract of rabbits will then be absorbed into the blood circulation. Both subcutaneous injection and oral feeding methods were used in order to hasten the development of atherosclerosis in the experimental rabbits in the limited time period.

Rabbits were used in this study rather than other animals because rabbits share several similar aspects of lipoprotein metabolism with humans except for the deficiency in hepatic lipase. On the other hand, rats and mice are not suitable to induce atherosclerosis. Rats are atherosclerosis-resistant species and HDL is the major plasma cholesterol carrier of rats. High HDL level and low cholesterol absorption rate contribute to the inappropriate use of mice in this study. Egg yolk had been used in this study in preparation of the high cholesterol diet that was given to the experimental rabbits to induce atherosclerosis. This is due to the fact that egg yolk contains about two-thirds lipid on a dry-weight basis. About one-third of the lipid is phospholipids and two-third is triacylglycerols. Cholesterol makes up a few percentage of total lipid.

The present study shows that oral feeding of Alternanthera sessilis ethanolic extract in the treatment group rabbits reduced the triglyceride level by 82% whereas untreated control group declined by only 66.4%. Besides, the results show that Alternanthera sessilis has potential in increasing the HDL level. However, the mechanism of these effects is unknown. It may be probably due to the anti-oxidant activity of the herb which can prevent the oxidation of LDL and cholesterol.

Changes therein and other uses will occur to those skilled in the art which are encompassed within the scope of the invention as defined by the scope of the claims.

1. A composition for lowering plasma triglyceride levels and/or increasing plasma high density lipoprotein levels upon administration towards a subject, comprising a polar extract of plant parts of Alternanthera sessilis.

2. The composition according to claim 1, wherein the administration is via oral ingestion.

3. The composition according to claim 1, wherein the polar extract is extracted via a solvent selected from the group consisting of water, methanol, isopropanol, acetonitrile and mixtures thereof.

4. The composition according to claim 1, wherein the plant parts are leaves and/or foliages.

5. A method for lowering plasma triglyceride levels in a subject, comprising orally administering a polar extract of the plant Alternanthera sessilis to the subject.

6. A method for increasing plasma high density lipoprotein levels in a subject, comprising orally administering a polar extract of the plant Alternanthera sessilis to the subject.

7. The method according to claim 5, wherein the polar extract is extracted via a solvent selected from the group consisting of water, methanol, isopropanol, acetonitrile and mixtures thereof.

8. The method according to claim 6, wherein the polar extract is extracted via a solvent selected from the group consisting of water, methanol, isopropanol, acetonitrile and mixtures thereof.
